(19) United States
(12) Patent Application Publication
(10) Pub. No.: US 2010/0022471 A1
(43) Pub. Date: Jan. 28, 2010

(54) Oral Moisturizer for Alleviating Dry Mouth

(75) Inventors: Paul H. Hanifl, Cary, IL (US); Greg Davis, Crystal Lake, IL (US); Jodi Balbinot, Cary, IL (US); Lori Pokorsky-Loy, Aledo, TX (US)

Correspondence Address: MILLER, MATTHIAS & HULL, ONE NORTH FRANKLIN STREET, SUITE 2350, CHICAGO, IL, 60606, US

(73) Assignee: SAGE PRODUCTS INC., Cary, IL (US)

(21) Appl. No.: 12/178,212

(22) Filed: Jul. 23, 2008

(51) Int. Cl. A61K 31/726; A61P 27/00

(52) U.S. Cl. 514/54

(57) ABSTRACT

An oral moisturizer is disclosed to provide relief from dry mouth. The moisturizer may be provided in different flavors that are palatable, with clinical benefits such as anti-nausea, appetite stimulant, etc., in addition to relief from the deteriorating dental effects of dry mouth. One disclosed oral moisturizer includes glycerin, xylitol, sodium hyaluronate, cetylpyridinium chloride, polysorbate surfactants and a palatable flavor.

### BACKGROUND

1. Technical Field

Compositions for the relief of xerostomia or “dry mouth” are disclosed that are useful for the treatment of disorders causing this condition, such as Sjögrens patients, oncology patients or patients suffering from dry mouth caused by one or more medications.

2. Description of the Related Art

Xerostomia, or “dry mouth,” is a symptomatic manifestation caused by a decrease in the activity of the salivary glands. Dry mouth can be a temporary condition caused by stress, infection of the salivary gland, or the use of certain drugs including anticholinergics, diuretics, antihistamines, clonidine, levodopa, methyldopa, and tricyclic antidepressants. Dry mouth can also be a permanent condition of an unknown origin.

Dry mouth has also been associated with Sjögrens syndrome, systemic sclerosis, and with oncology treatments of all kinds including, but not limited to radiation therapy of the mouth, neck and head during treatment of mouth cancer. Chronic dry mouth leads to difficulty and soreness in swallowing, speaking, and dry mouth can interfere with taste sensation. Because saliva has anti-microbial properties and a general cleaning function, dry mouth also leads to tooth decay. Dry mouth is a widespread problem amongst senior citizens as salivary secretion can decrease with age causing problems such as gingivitis, cavity formation, and the appearance of canker sores.

Dry mouth is currently treated with mouth rinses, topical applications, salivary substitutes, or salivary stimulants such as sugarless candies and chewing gum. Some saliva stimulants are commercially available. One common treatment for treating dry mouth is spraying the inside of the mouth or rubbing the interior of the mouth with a saliva substitute. However, currently available products require frequent application, and thus are cumbersome for the patient or hospital personnel. Further, most products are unpalatable and patients have a tendency to not use them with sufficient frequency.

### SUMMARY OF THE DISCLOSURE

An improved oral moisturizer is disclosed to provide relief from dry mouth, for individuals with chronic dysfunctions of the natural salivary process (Sjögrens patients, cancer patients, etc.) or patients that require long-term use of medication that causes dry mouth as a side effect. The disclosed moisturizer may be provided in different flavors that are palatable, with potential clinical benefits such as anti-nausea, appetite stimulant, etc.

One disclosed oral moisturizer comprises: glycerin; xylitol; sodium hyaluronate; and cetylpyridinium chloride.

In a refinement, the oral moisturizer further comprises a surfactant. In a further refinement of this concept, the surfactant comprises at least one polyoxyethylene sorbitan monooleate. In still another refinement of this concept, polyoxyethylene sorbitan monooleate comprises a combination of polyoxyethylene (20) sorbitan monooleate and polyoxyethylene (80) sorbitan monooleate.

In another refinement, the oral moisturizer comprises potassium sorbate.

In another refinement, the oral moisturizer provides a flavor of spearmint, cinnamon or mandarin orange. Other flavors and flavor combinations are available and will be apparent to those skilled in the art. The flavors spearmint, cinnamon and mandarin orange, however, have been found to be particularly palatable, which encourages frequent use of the product, and improves the dental health of the patient.

In another refinement, the glycerin is present in an amount ranging from about 50 to about 75 wt %, the xylitol is present in amount ranging from about 2 to about 10 wt %, the sodium hyaluronate is present in an amount ranging from about 0.01 to about 1.0 wt %, and the cetylpyridinium is present in an amount ranging from about 0.01 to about 0.04 wt %.

In another refinement, the oral moisturizer includes water in an amount ranging from about 50 wt % to about 75 wt %.

In another refinement, the polyoxyethylene (20) sorbitan monooleate, and polyoxyethylene (80) sorbitan monooleate are each present in an amount ranging from about 0.10 to about 3.0 wt %.

Other advantages and features will be apparent from the following detailed description when read in conjunction with the attached drawings.

### DETAILED DESCRIPTION OF THE PRESENTLY PREFERRED EMBODIMENTS

Improved formulations for relief from xerostomia or dry mouth are disclosed. The disclosed formulations are particularly useful for patients suffering from disease that adversely affect the natural salivary process. Such diseases include, but are not limited to Sjögrens syndrome, various forms of cancer, or patients needing specific medications that can cause dry mouth as a side effect. Such medications include anticholinergics, diuretics, antihistamines, clonidine, levodopa, methyldopa, tricyclic antidepressants, medications for the treatment of attention deficit disorder and appetite suppressants.

Two humectants are utilized in the disclosed moisturizers. First, sodium hyaluronate (a.k.a. hyaluron, hyaluronic acid or hyaluronate) is utilized in relatively small amounts of 3 wt % or less, more preferably 2 wt % or less in most preferably in a range of from about 0.01 to about 1.0 wt %. One preferred formulation includes 0.025 wt % sodium hyaluronate.

Sodium hyaluronate (a.k.a. hyaluronic acid, hyaluron or hyaluronate) is a particularly good moisturizer, lubricant and humectant. Hyaluronate is naturally present in saliva and other areas of the body.

Specifically, hyaluronate is a non-sulfated glycosaminoglycan distributed widely throughout connective, epithelial, and neural tissues of the human body and is a part of the extracelluar matrix. Hyaluronate is therefore well suited to biomedical applications. Humans degrade and synthesize hyaluronate continuously. Hyaluronate is an important component of articular cartilage, where it is present as a cell coating (chondrocyte). Hyaluronate is also found in human skin, where it is believed to function in tissue repair. While it is abundant in extracellular matrices, hyaluronate also contributes to tissue hydrodynamics, movement and proliferation of cells, and participates in a number of cell surface receptor interactions. The chemical structure of hyaluronate is shown below:

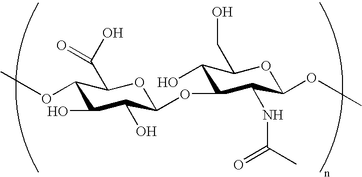

Hyaluronate is stable in part because of the stereochemistry of its component disaccharides. The bulky groups on each sugar molecule are in sterically favored positions, whereas the smaller hydrogens assume the less-favorable axial positions. The term hyaluronate refers to the conjugate base of hyaluronic acid. Because the molecule typically exists in vivo in its polyanionic form, it is commonly referred to as hyaluron. The terms of sodium hyaluronate, hyaluronate, hyaluronic acid and hyaluron will be used interchangeably herein. Aloe Vera gel could be used with the sodium hyaluronate.

The second humectant, glycerin is commonly referred to as glycerol or glycerine and these terms will be used interchangeably herein. Glycerin is a colorless, odorless, viscous liquid that is widely used in pharmaceutical formulations. Glycerin is a sugar alcohol, is sweet-tasting and of low toxicity, thereby making it an ideal humectant for the disclosed oral moisturizer. Glycerin has three hydrophilic alcoholic hydroxyl groups that are responsible for its solubility in water and its hygroscopic nature. The glycerin structure is a as follows:

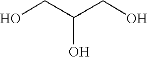

Glycerin is a precursor for synthesis of triacylglycerols and of phospholipids in the liver and adipose tissue. When the body uses stored fat as a source of energy, glycerol and fatty acids are released into the bloodstream. The glycerol component can be converted to glucose by the liver and provides energy for cellular metabolism.

Glycerin can be used in the disclosed oral moisturizers in a relatively wide range. Less than 5 wt % most likely does not provide enough humectant properties to be effective, especially with chronic cases. More than 40 wt % may render the formulation too viscous, making the moisturizer difficult to apply and therefore undesirable to the patient. Preferably, glycerin is present in an amount ranging from about 5 wt % to about 30 wt %. In one preferred embodiment, glycerin is present in amount of about 25%. Propylene glycol may be used in addition to glycerin.

Xylitol is a five-carbon sugar alcohol with the following structure that is used as a sugar substitute and is therefore palatable.

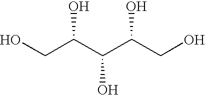

Xylitol is a naturally occurring sweetener found in the fibers of many fruits and vegetables, including various berries, corn husks, oats, and mushrooms. Xylitol is roughly as sweet as sucrose but with only two-thirds the food energy. Xylitol does not promote tooth decay. In fact, xylitol may aid in repairing minor cavities caused by dental caries and also has a plaque-reducing or bacterial biofilm inhibiting effect.

The amount of xylitol used in the disclosed formulation may also vary widely from about 2 wt % to about 10 wt %. In a preferred embodiment, xylitol is present in a range from about 5 wt % to about 8 wt %, most preferably about 7 wt %. Suitable alternative sweeteners include, but are not limited to: sorbitol, mannitol, maltitol, sodium saccharin, sucralose, and combinations thereof.

Cetylpyridinium chloride (CPC) is a cationic quaternary ammonium compound used as a preservative and has both antiseptic and anti-microbial properties.

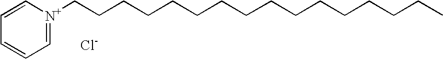

CPC is also effective in reducing dental plaque and reducing gingivitis. It is anticipated that the combination of xylitol and CPC will provide improved bacterial biofilm inhibition or plaque resistance and therefore the combination of xylitol and the CPC will render the disclosed oral moisturizer suitable for long-term use.

Surfactants are preferably included for solubilizing purposes and to aid in maintaining the moisturizer on the surfaces of the mouth. Preferred surfactants include polysorbates. Polysorbates are derived from polyethylene glycol (PEG) or PEG-ylated sorbitan (a derivative of sorbitol) esterified with fatty acids. Two preferred polysorbate surfactants include pₒlyₒxyethylene (20) sorbitan monooleate and polyoxyethylene (80) sorbitan monooleate. The numbers 20, 80 following the polyoxyethylene term refers to the total number of oxyethylene —(CH₂CH₂O)— groups (w+x+y+z) found in the molecule:

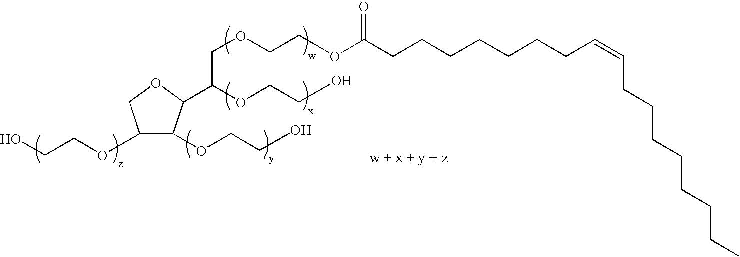

The above surfactants are sold under the TWEEN® 20 and TWEEN® 80 trademarks (http://www.icinorthamerica.com/). Substitutes for polysorbate surfactants include but are not limited to PEG40 hydrogenated castor oil, PEG 40 sorbitan diisostearate, Poloxamer 407, a polyethylene-polypropylene polymer and combinations thereof.

The relative amounts of polysorbate 20 and polysorbate 80 can vary widely from about 0.10 wt % to about 5 wt %, more preferably from about 0.10 wt % to about 3 wt %. In a preferred embodiment, polysorbate 20 and polysorbate 80 are both present in amounts of about 0.20 wt %.

Potassium sorbate is also used as a preservative.

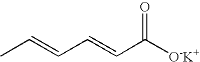

The amount of potassium sorbate is relatively low, less than 1 wt %, more preferably less than 0.5 wt %, more preferably in the range of from about 0.1 wt % to about 0.2 wt %. In a preferred embodiment, the potassium sorbate is present in amount of about 0.20 wt %. Suitable alternatives to potassium sorbate include, but are not limited to benzoic acid, sodium benzoate or combinations thereof, methylparaben, and propylparaben.

One key advantage of the disclosed oral moisturizers is the palatability. An unpalatable formulation will not be used by the patient, and therefore adversely affect the patient's dental health. Spearmint, cinnamon and mandarin (orange) flavors have been developed that patients find both palatable and enjoyable, which encourages frequent use. The spearmint flavoring can range up to about 5 wt %, more preferably in the range of from about 1 wt % to about 3 wt %. The cinnamon flavoring can also range up to 1 wt %, more preferably in the range of 0.1 wt % to about 0.5 wt %. The mandarin orange flavoring similarly can range up to about 1 wt %, more preferably from about 0.1 wt % to about 0.5 wt %. Other flavorings can be utilized and the relative amounts will depend upon the concentrations provided by the suppliers. These flavors and xylitol have been found to create a very palatable if not pleasing oral moisturizer that patients with chronic dry mouth will use.

Other suitable flavor combinations include, but are not limited to lemon, grapefruit, lime, citrus, tangerine, lemonade, ginger, ginger-lemon, cranberry, oregano, basil, sage, rosemary, bacon, cheese, beef, or green mint tea.

While only certain embodiments have been set forth, alternatives and modifications will be apparent from the above description to those skilled in the art. These and other alternatives are considered equivalents and within the spirit and scope of this disclosure and the appended claims.

What is claimed:

1. An oral moisturizer comprising:
glycerin;
xylitol;
sodium hyaluronate; and
cetylpyridinium chloride.

2. The oral moisturizer of claim 1 further comprising a surfactant.

3. The oral moisturizer of claim 2 wherein the surfactant comprises at least one polyoxyethylene sorbitan monooleate.

4. The oral moisturizer of claim 3 wherein the polyoxyethylene sorbitan monooleate comprises:
polyoxyethylene (20) sorbitan monooleate; and
polyoxyethylene (80) sorbitan monooleate.

5. The oral moisturizer of claim 3 wherein the polyoxyethylene sorbitan monooleate is selected from the group consisting of:
polyoxyethylene (20) sorbitan monooleate; and
polyoxyethylene (80) sorbitan monooleate.

6. The oral moisturizer of claim 1 further comprising potassium sorbate.

7. The oral moisturizer of claim 1 further comprising a flavor selected from the group consisting of:
spearmint flavor;
cinnamon flavor; and
mandarin flavor.

8. The oral moisturizer of claim 1 wherein the glycerin is present in an amount ranging from about 5 to about 30 wt %, the xylitol is present in amount ranging from about two to about 10 wt %, the sodium hyaluronate is present in an amount ranging from about 0.01 to about 1.0 wt %, and the cetylpyridinium is present in an amount ranging from about 0.01 to about 0.04 wt %.

9. The oral moisturizer of claim 1 further comprising water in an amount ranging from about 50 to about 75 wt %.

10. The oral moisturizer of claim 8 further comprising water in an amount ranging from about 50 to about 75 wt %.

11. The oral moisturizer of claim 5 wherein the polyoxyethylene (20) sorbitan monooleate, and polyoxyethylene (80) sorbitan monooleate are each present in an amount ranging from about 0.10 to about 3.0 wt %.

12. An oral moisturizer comprising:
glycerin;
xylitol;
sodium hyaluronate;
cetylpyridinium chloride;
a surfactant; and
a flavor selected from the group consisting of spearmint, cinnamon and mandarin.

13. The oral moisturizer of claim 12 wherein the surfactant comprises at least one polyoxyethylene sorbitan monooleate.

14. The oral moisturizer of claim 12 wherein the glycerin is present in an amount ranging from about 5 to about 30 wt %, the xylitol is present in amount ranging from about two to about 10 wt %, the sodium hyaluronate is present in an amount ranging from about 0.01 to about 1.0 wt %, and the cetylpyridinium is present in an amount ranging from about 0.01 to about 0.04 wt %.

15. The oral moisturizer of claim 12 further comprising water in an amount ranging from about 50 to about 75 wt %.

16. The oral moisturizer of claim 13 wherein the at least one polyoxyethylene sorbitan monooleate comprises polyoxyethylene (20) sorbitan monooleate and polyoxyethylene (80) sorbitan monooleate, which are each present in an amount ranging from about 0.10 to about 3.0 wt %.

17. A palatable oral moisturizer for use by patients suffering from dry mouth, the oral moisturizer comprising:
at least 50 wt % water;
at least 5 wt % glycerin;
at least 2 wt % xylitol;
less than 2 wt % sodium hyaluronate;
less than 0.08 wt % cetylpyridinium chloride;
less than 6 wt % polysorbate surfactant; and
a flavor selected from the group consisting of spearmint, cinnamon and mandarin.

18. The oral moisturizer of claim 17 wherein the water is present in an amount ranging from about 50 to about 75 wt %, the glycerin is present in an amount ranging from about 5 to about 30 wt %, the xylitol is present in amount ranging from about two to about 10 wt %, the sodium hyaluronate is present in an amount ranging from about 0.01 to about 1.0 wt %, and the cetylpyridinium is present in an amount ranging from about 0.01 to about 0.04 wt %.

19. The oral moisturizer of claim 18 wherein the polysorbate surfactant comprises polyoxyethylene (20) sorbitan monooleate and polyoxyethylene (80) sorbitan monooleate, which are each present in an amount ranging from about 0.10 to about 3.0 wt %.
